(19) United States
(12) Patent Application Publication
(10) Pub. No.: US 2013/0178762 A1
(43) Pub. Date: Jul. 11, 2013

(54) COLOR-CODED RINGS FOR DETERMINING DEGREE OF CERVICAL DILATATION

(71) Applicants: Asha Krishna PRATINIDHI, Kolhapur (IN); Krishna Shridharrao PATIL, Pune (IN); Aparna Nishikant SHROTRI, Pune (IN); Hemangi Parag KALE, Solon, OH (US)

(72) Inventors: Asha Krishna PRATINIDHI, Kolhapur (IN); Krishna Shridharrao PATIL, Pune (IN); Aparna Nishikant SHROTRI, Pune (IN); Hemangi Parag KALE, Solon, OH (US)

(73) Assignee: KRISHNA INSTITUTE OF MEDICAL SCIENCES UNIVERSITY, Karad (IN)

(21) Appl. No.: 13/343,817

(22) Filed: Jan. 5, 2012

(51) Int. Cl. A61B 5/103

(52) U.S. Cl. 600/588

(57) ABSTRACT

A set of color coded rings consisting of one subset of even numbers and other subset of odd numbers together is indicative of serially increasing cervical dilatation from 4 cm. to 9 cm. Vaginal examination with all aseptic precautions is to be carried out every 2 to 4 hours for each delivering woman. Size and color of the ring corresponding to actual cervical dilatation is to be identified. Next ring of the same color indicates expected cervical dilatation at the end of every 2 hours. If the expected cervical dilatation is not reached within the stipulated period of time, it indicates slow progress of labor and need for intervention/referral. It is an alternative for cervicograph which is easy to understand and use by clinical as well as para-clinical personnel during delivery.

### BACKGROUND OF THE INVENTION

The present invention relates to the field of medical sciences and more particularly to the field of obstetrics wherein a set of color coded rings of two colors with increasing diameter from 4 cm, 6 cm, 10 cm of one color and 5 cm, 7 cm, 9 cm of another color is prepared with an objective of giving visual impressions of existing and expected sizes of the cervix during the active phase of the first stage of labor. Briefly, the invention relates to color coded rings designed to allow nursing, paramedical and clinical staff to identify and diagnose delayed labor.

Reduction in neonatal mortality is a challenging task in the developing countries. Two important causes of perinatal and neonatal mortality are birth asphyxia & low birth weight. Prolonged or obstructed labor can lead to fetal asphyxia which may account for neonatal morbidity/mortality. However, neonatal morbidity/mortality can be, to some extent, reduced by proper monitoring of labor. Also, numerous women all over the world die annually due to complications of pregnancy and child birth. Most of these deaths are also due to prolonged and obstructed labor and its complications, such as sepsis, dehydration and uterine rupture. Most of these deaths are preventable.

Partographic monitoring of labor is the most suitable method available at present and is the method recommended by the World Health Organization (“WHO”). Partography is a graphical method of recording the events in or during labor. A partograph is displayed in the labor and delivery room for easy and quick assessment by personnel of labor progress and timing of management decisions. Partography was designed to predict and indentify labors that may be prolonged.

Although palpatory observations of cervical dilatation were first made by Ebermair in 1816. In 1954, Friedman introduced the concept of a graphic record of the progress of labor, and this concept was further simplified by Philpot in 1972. The WHO has recommended the use of partography for the identification and referral of cases of prolonged labor. The WHO, as part of an initiative aimed at reducing maternal mortality and morbidity, has recommended the use of partographs since 1988 for labor management in developing countries, especially in peripheral health centers. Essential features of these partographs are a cervicograph, which comprises the latent and active phase of labor and incorporates the action and alert lines in the active phase of labor and columns for fetal and maternal monitoring and that of quality of uterine contractions, as in the WHO partograph. This is done by observing the rate of labor progress using the most objective index, which is cervical dilatation, plotted against time, and the rate of descent of the presenting part. These observations will assist in timing management decisions. Any labor that progresses at any rate less than this needs reassessment and some form of intervention to ensure normal progress. The use of the partograph is to be done in following manner.
Patients are admitted into the labor ward and put on a partograph once the active phase of labor is diagnosed and a vaginal examination is performed every four hours.
During the active phase of labor, cervical dilatation of >1 cm/hour is expected.
The quality of uterine contractions, fetal heart rate and maternal vital signs are monitored and evaluated.
The patient is transferred from a peripheral unit to a central unit if there is a prolonged latent phase or if the active phase labor is progressing at less than 1 cm/hour; i.e., if the cervicogram falls to the right of the alert line.
At the central unit, reassessment is made, and appropriate interventions are carried out in terms of rupture of membranes, rehydration, antibiotics, analgesia, and caesarean section, as the case may be.

Partography, though recommended by the WHO, is not always used in practice, as the plotting of cervical dilatation against time is found to be a difficult task by attending physician and paramedical workers. In India, for example, partography is not routinely used in maternity units.

### BRIEF SUMMARY OF THE INVENTION

Briefly stated, in one embodiment, the present invention is directed to a first subset of color coded rings and a second subset of color coded rings. A diameter of each ring of the first subset of color coded rings is an even number and a diameter of each ring of the second subset of color coded rings is an odd number. Together, the diameters are indicative of serially increasing cervical dilatation from 4 centimeters to 9 centimeters.

Briefly stated, in another embodiment, the present invention is directed to a method of identifying delayed labor. The method comprises providing a first subset of color coded rings and a second subset of color coded rings, a diameter of each ring of the first subset of color coded rings being an even number and a diameter of each ring of the second subset of color coded rings being an odd number, together the diameters being indicative of serially increasing cervical dilatation. The method further comprises determining by a first vaginal examination of the actual initial cervical dilatation of a patient, determining whether the first subset of color coded rings or the second subset of color coded rings is to be used, performing a second vaginal examination of the patient after a duration of two hours has elapsed, identifying the ring which represents the expected cervical dilatation during the time of the second vaginal examination, and determining if the actual cervical dilatation during the second vaginal examination corresponds the expected cervical dilatation.

### BRIEF DESCRIPTION OF THE SEVERAL VIEWS OF THE DRAWINGS

The foregoing summary, as well as the following detailed description of the invention, will be better understood when read in conjunction with the appended drawings. For the purpose of illustrating the invention, there are shown in the drawings an embodiment which is presently preferred. It should be understood, however, that the invention is not limited to the precise arrangements shown.

In the drawings:

FIG. 1 depicts a first subset of 4, 6 and 8 cm rings of one color and a second subset of 5, 7 and 9 cm rings of another color in accordance with a preferred embodiment of the present invention; and

FIG. 2 depicts a chart of 4, 6 and 8 cm rings of one color and a second subset of 5, 7 and 9 cm rings of another color in accordance with another preferred embodiment of the present invention.

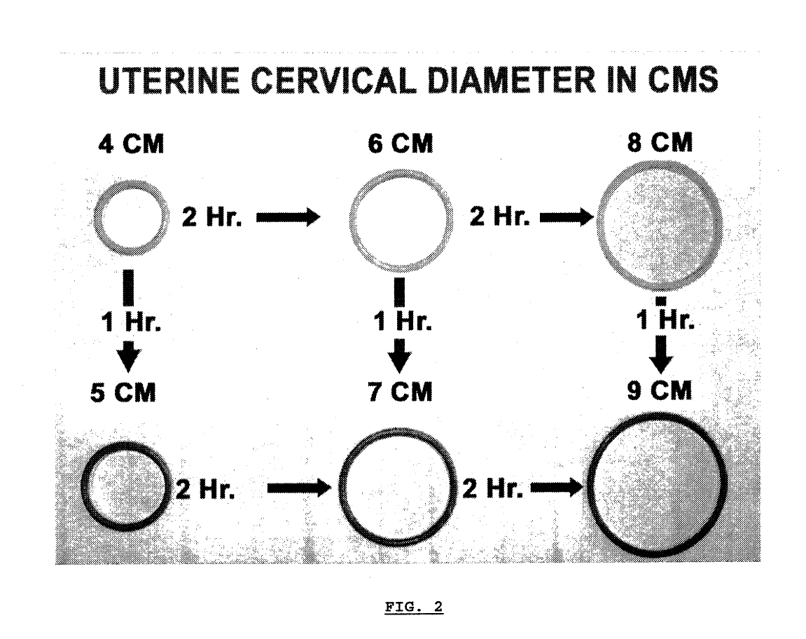

### DETAILED DESCRIPTION OF THE INVENTION

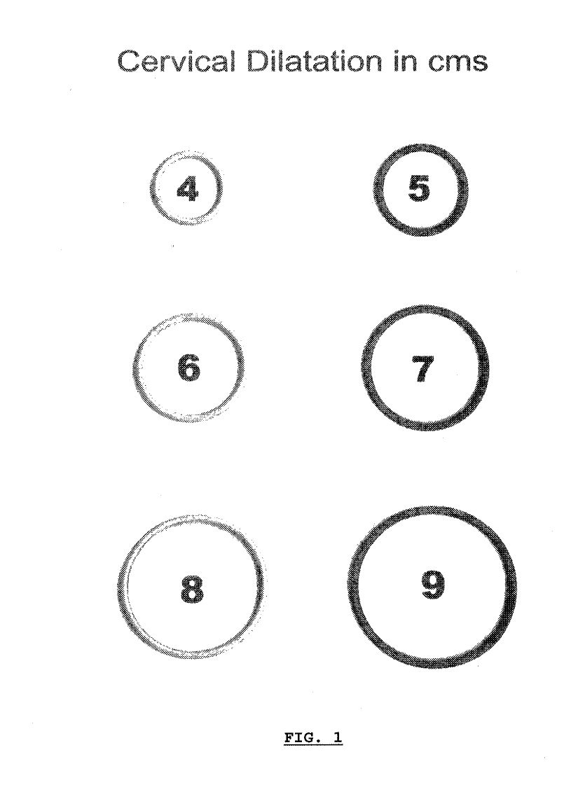

A preferred embodiment of the present invention relates to color-coded rings, as shown in FIG. 1, which can be used as a tool to translate partographic principles into a simpler and easier to understand methodology for determining if a labor/delivery is being prolonged. Deliveries are to be monitored by the color-coded rings for judging satisfactory progress of labor during the first stage of labor. Internal assessment is generally done at two to four hour intervals. The set of color-coded rings according to a preferred embodiment of the present invention is devised in such a way that the rings would assist personnel in identifying unusual delays in labor at the four and two hour interval examinations. The minimum expected cervical dilatation rate of 1 cm/hour during the active phase is depicted in the preferred embodiment of the present invention shown in FIG. 1.

It is a common observation that sizable proportions of primis and multies come for delivery at rural hospitals and primary health facilities at or more than 6 centimeter cervical dilatation stage. In these cases, assessment of the cervical dilatation after two hours may be more useful as the time taken by the woman to reach this sage before admission to the health facility is not known. In routine practice, in rural areas, vaginal examination is often done after two hours due to change in shifts of nurses, pressure from relatives and to know progress of labor in high risk cases. The color-coded rings according to a preferred embodiment of the present invention together are capable of indicating total delay in progress of labor at an hourly interval. If a second vaginal examination undertaken at the end of further two hours (i.e., after a total four hours of labor) indicates delayed labor, personnel can then refer the patient to a hospital with facilities for comprehensive emergency obstetric care.

Cervicographic assessment of labor relates to the active phase of the first stage of labor. It is usually accepted that the active stage of labor starts at a cervical dilatation of 4 centimeters, which is when an initial assessment is done. A cervicograph is based on the principle that after commencement of the active stage of labor, it is expected that the minimum rate of cervical dilatation will be approximately 1 cm/hour. As per WHO guidelines, a second vaginal examination is to be undertaken four hours after the initial assessment.

The color-coded rings of the present invention are devised using these same principles. All rings of the subsets of color-coded rings according to a preferred embodiment of the present invention have a diameter from 4 cm. to 9 cm. One color is preferably used for rings having a 4, 6, or 8 cm. diameter and another color is preferably used for rings having a 5, 7, or 9 cm. diameter.

During vaginal examination, birth attendants can obtain his/her own visual impression about cervical dilatation in terms of the distance between two fingers, which is mentally converted into centimeters before using a partogram. Instead, the color-coded rings according to a preferred embodiment of the present invention are more objective in giving a visual impression of existing and expected sizes of the cervix reducing inter-observer variation. The color-coded rings according to a preferred embodiment of the present invention, which is based on the same principles as cervicographs, are thus easy to understand and use. Hence, the possibility of their use for the identification of delayed labor can be ensured.

Vaginal examinations, with all aseptic precautions, are to be done every four hours and, if required, every two hours. The color-coded rings according to a preferred embodiment of the present invention are prepared in such a way that there is a 4 centimeter difference between the smallest and the largest ring of each subset of rings of the same color. The smallest ring of each subset corresponds with the initial cervical dilatation (i.e., of 4 or 5 cm) and the largest ring of each subset corresponds with the expected cervical dilatation at the end of four hours of a normal labor (i.e., of 8 or 9 cm). The middle sized ring of each subset of the same color indicates the minimum cervical dilatation expected at the end of two hours of a normal labor. Preferably, the first and second subsets of color-coded rings are mounted or drawn on a hard board, which is preferably hung on a wall for ready reference of the birth attendants.

After vaginal examination the birth attendant translates the observed cervical dilatation into the small, medium and large sized rings of a particular color and the corresponding cervical dilatation of 4 to 9 cm, respectively, as shown in FIG. 1. If the initial cervical dilatation is in even numbers (e.g., 4 cm), after two hours, the middle ring of the even number subset represents the expected minimum cervical dilatation. After four hours, the largest ring of the even number subset represents the expected minimum cervical dilatation. If the initial cervical dilatation is an odd numbers (e.g., 5 cm), the expected minimum cervical dilatation after two hours of labor is represented by the medium sized ring of the odd number subset, and after four hours, the expected minimum cervical dilatation is represented by the largest ring of the odd number subset. If an expected dilatation size is not reached, that indicates that the labor is progressing slowly and that there is likely a need for intervention.

If these subsets of color-coded rings are given to labor and delivery personnel, such as nurses, midwives, doctors and the like, particularly at rural hospitals and primary care facilities, deliveries can be very easily monitored for early diagnoses of prolonged labor. The color coded rings according to a preferred embodiment of the present invention represent the first time that the rate of cervical dilatation is translated into an easily understandable concept, which does not require any complicated, graphical presentation or maintaining of a graphical record. Instead, only the mention of color and size of the first and second subsets of rings is sufficient to indicate the progress of labor.

The additional advantages of the set of rings according to a preferred embodiment of the present invention include the following:

1) The rings can replace cervicographs.

2) The rings are easy to understand.

3) The set of rings is an easy application of the principles of cervicography.

4) The rings give better visual impression of the potential sizes of a cervix and the objective criteria of cervical dilatation. Being more objective gives precise size of actual and expected cervical dilatation, and judgment is more accurate as compared to conversion of subjective feeling of dilatation in fingers and corresponding size in cm as per impression of the birth attendant.

5) The visual presentation of cervical dilatation helps in explaining the progress of labor, actual and expected cervical dilatation in relation to time to patients and their relatives.

6) The rings are economical. Only one of each of the first and second subsets of rings for one delivery room is sufficient, as compared to partographs which are needed on a one per patient basis. There is no need of individual subsets for each delivering woman because the rings need not actually contact the patient.

7) The use of the rings is only for visual impressions. There is no need of handling the rings during delivery and, hence no need of sterilization and hence no risk of infection.

8) There is no need of plotting a graph and, hence the rings save time for birth attendants for judging slow progress of labor.

It will be appreciated by those skilled in the art that changes could be made to the embodiments described above without departing from the broad inventive concept thereof. It is understood, therefore, that this invention is not limited to the particular embodiments disclosed, but it is intended to cover modifications within the spirit and scope of the present invention as defined by the appended claims.

We claim:

1. A first subset of color coded rings and a second subset of color coded rings, a diameter of each ring of the first subset of color coded rings being an even number and a diameter of each ring of the second subset of color coded rings being an odd number, together the diameters being indicative of serially increasing cervical dilatation from 4 centimeters to 9 centimeters.

2. A method of teaching birth attendants how to accurately assess cervical dilatation and progress of labor using the first and second subsets of color coded rings according to claim 1.

3. A method of evaluating a level of skill of birth attendants after the birth attendants have been taught how to accurately assess cervical dilatation and progress of labor according to claim 2.

4. A method of identifying delayed labor, the method comprising:
providing a first subset of color coded rings and a second subset of color coded rings, a diameter of each ring of the first subset of color coded rings being an even number and a diameter of each ring of the second subset of color coded rings being an odd number, together the diameters being indicative of serially increasing cervical dilatation;
determining by a first vaginal examination actual initial cervical dilatation of a patient;
determining whether the first subset of color coded rings or the second subset of color coded rings is to be used;
performing a second vaginal examination of the patient after a duration of two hours has elapsed;
identifying the ring which represents the expected cervical dilatation during the time of the second vaginal examination;
determining if the actual cervical dilatation during the second vaginal examination corresponds the expected cervical dilatation.

5. The method according to claim 4, wherein if the expected cervical dilatation is not reached, delayed progress of labor is indicated.
